(19) United States
(12) Patent Application Publication
(10) Pub. No.: US 2021/0060363 A1
(43) Pub. Date: Mar. 4, 2021

(54) K9 Quick-Clip Oxygen Muzzle

(71) Applicant: Thomas Kenney Barrett, JR., Midliothian, VA (US)

(72) Inventor: Thomas Kenney Barrett, JR., Midliothian, VA (US)

(21) Appl. No.: 16/292,558

(22) Filed: Jul. 25, 2019

Related U.S. Application Data
(60) Provisional application No. 62/761,047, filed on Mar. 5, 2018.

(51) Int. Cl. A62B 18/06; A61M 16/10; A61M 16/08; A61M 16/06; A62B 9/04; A01K 25/00

(52) U.S. Cl. CPC A62B 18/06; A61M 16/1005; A61M 16/0816; A61M 2202/0208; A61M 16/0605; A62B 9/04; A01K 25/00; A61M 16/0875

(57) ABSTRACT

$ dual-purpose muzzle is capable of protecting the canine handler from a bite while providing lifesaving oxygen to a canine in distress. The muzzle is designed to clip behind the head like a soft muzzle while delivering needed oxygen with its internal oxygen tubing and specially designed port for quick hook up. The port allows for any standard EMS tubing to connect to the muzzle. This allows for a continuous flow of oxygen directed to the snout and mouth of the canine allowing the handler to be hands free to address any other emergencies.

### CROSS REFERENCE TO RELATED APPLICATION

This application claims benefit of the U.S. Provisional Application 62/761,047 filed on Mar. 5, 2018 entitled “K9 Quick-Clip Oxygen Muzzle”.

### FIELD OF THE INVENTION

The present invention relates to oxygen therapy in the field for canines. As canines have become more involved in military, law enforcement, and search and rescue more up to date medical procedures have been implemented to deal with more frequent emergencies. As humans do, canines also need oxygen to survive. For the lack of rapid oxygen therapy to canines in the field, we have developed a dual purpose muzzle capable of protecting the canine handler from a bite while providing lifesaving oxygen to a canine in distress.

The muzzle is designed to clip behind the head like a soft muzzle while delivering needed oxygen with its internal oxygen tubing and specially designed O2 Port for quick hook up. The port allows for any standard EMS tubing to connect to the muzzle. There will be different locations of the port, front of muzzle or on the side strap, depending on preference.

This allows for a continuous flow of oxygen directed to the snout and mouth of the canine allowing the handler to be hands free to address any other emergencies.

The O2 Muzzle is quick and easy to put on and made of highly durable webbing making it lightweight and tactical.

The full harness system can be utilized in most austere environments as the canine will be carrying his own oxygen. The system is lightweight and fully protected. All oxygen lines and hookups are encased in soft durable material suited for any outdoor environment.

### DESCRIPTION OF THE PRIOR ART

There is one known item used for oxygen therapy for canines in an urban environment and not in a working field environment. It is a hard plastic cone with a hook up. It is not compactable nor flexible.

### SUMMARY OF THE INVENTION

With the lack of means to provide proficient oxygen therapy in the field for working canines the present invention allows accessibility to oxygen therapy in remote areas where there otherwise may not be any. The soft flexible design of the muzzle allows for it to be folded or packed into a pocket ready for use if the need arises.

The invention allows for it to be re-used by cleaning with alcohol wipe or cleaning wipe not dangerous to the canine.

The soft flexible design of the muzzle allows for it to be folded or packed into a pocket ready for use if the need arises. Being lightweight makes it more pleasing to carry in austere environments.

The invention is also designed to work like a bite muzzle and protect the canine handler from a bite while still allowing ample room for the canine to pant. The durability of the material gives confidence of this.

### BRIEF DESCRIPTIONS OF THE DRAWINGS

FIG. 1A illustrates the front nose piece, top and bottom O2 delivery ports, soft mesh left side, both left and right internal O2 tubes inside rear straps.

FIG. 1B illustrates the rear straps where the oxygen port is located for easy hook up.

FIG. 2a illustrates the right side interior cross section showing quick-clip.

FIG. 2B illustrates an inside view at front nose piece with O₂ delivery port and tubing.

FIG. 2C illustrates an exterior front nose piece and straps.

FIG. 3A illustrates the outer tube protective cover and dual hook up O₂ port for full harness system.

FIG. 3B illustrates the Oxygen Tee connector for oxygen direction according to the present invention.

FIG. 4A illustrates the Full Neck Harness System with O₂ collar.

FIG. 4B illustrates from another angle the Full Neck Harness System on the canine.

FIG. 4C shows additional placement of Oxygen Port for hookup.

FIG. 5 illustrates the Full Harness System, neck, chest and back, each with O₂ carrying capability.

### DETAILED DESCRIPTION OF THE INVENTION

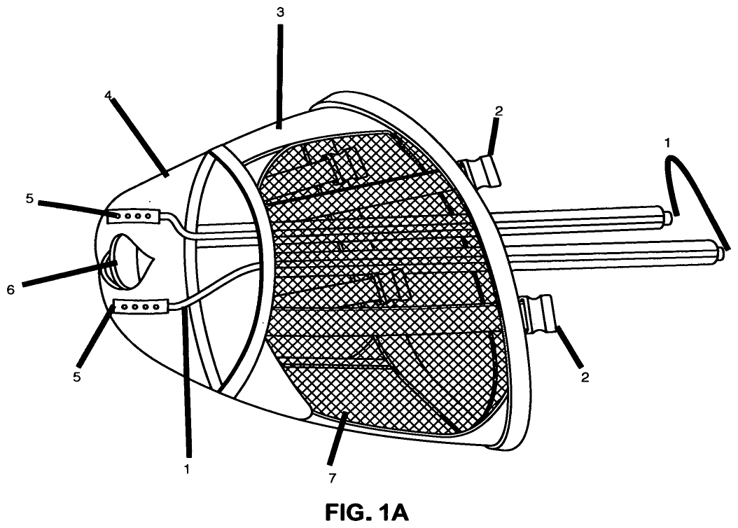

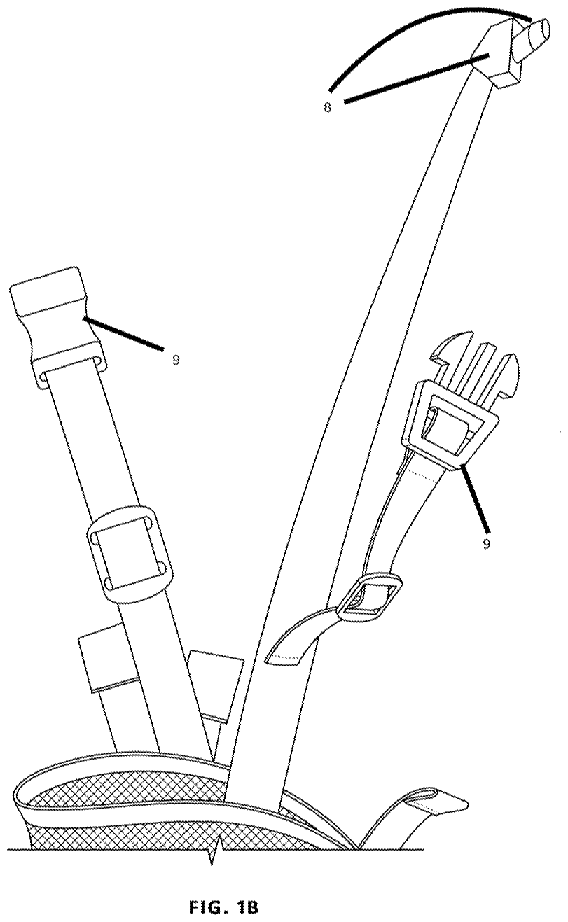

Referring to FIG. 1A, this shows the full frontal nose and left side lateral view. The rear straps contain the internal oxygen tubing (1) which is encased and protected in a soft reinforced webbing material which is able to be folded and put in a pocket. The tubing runs toward the nose piece exiting the oxygen ports (5) directly in front of the canines' snout allowing for maximum delivery. The J clips (2) shown on the outer rear ring are optional. The four J clips can be added on bungee straps to attach over a pre-existing muzzle by four points of contact keeping it in place. The Velcro flap on top (3) is able to loosen or tighten to adapt to different size canines preventing the need to carry multiple sizes. The solid flexible nose piece (4) helps with better oxygenation in the muzzle. The front nose has an optional front mounted Oxygen hook up allowing for a different configuration depending on the mission. The nose also allows for a front oxygen port to be put in (6) depending on what the need is.

Looking at FIG. 1B shows the rear straps with a heavy duty buckle which connects behind the canines head just like a soft muzzle. The rear integrated Oxygen Port (8) has easy hook up to any standard oxygen line. All tubing is encased in the flexible webbing for protection with industrial stitching. All clasps and buckles and straps are industrial stitched for durability.

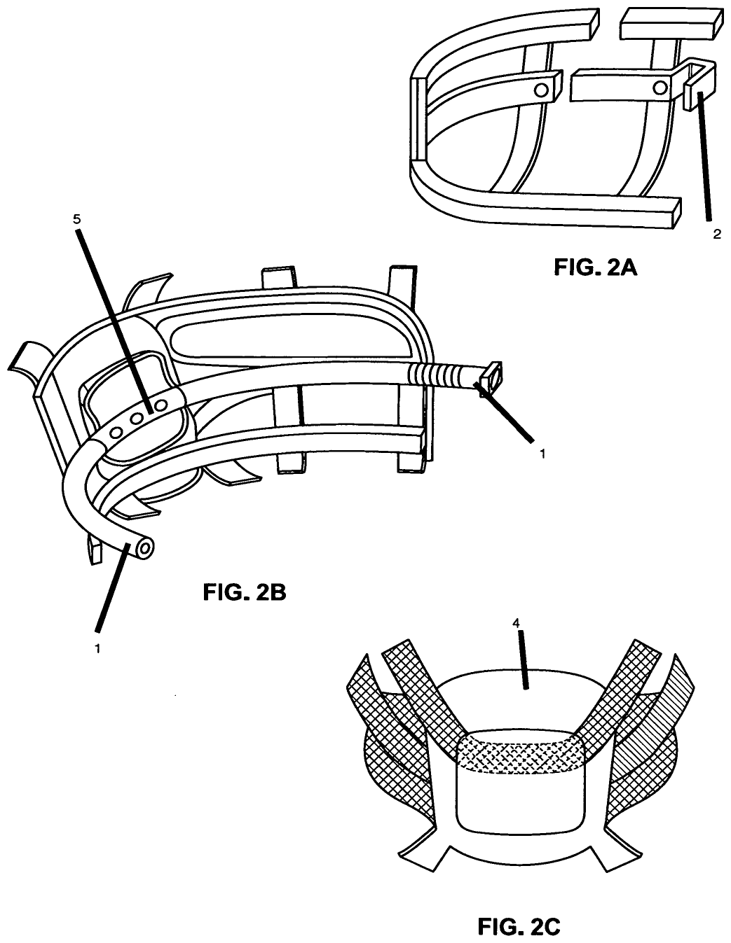

Referring to FIG. 2A, it shows the right internal design with J clip (2). FIG. 2B shows the internal oxygen tubing (1) on both sides which normally is encased in protective webbing. The oxygen delivery port (5) is directly in front blowing O2 into the canines nose and mouth. The soft solid nose piece (4) is flexible and durable.

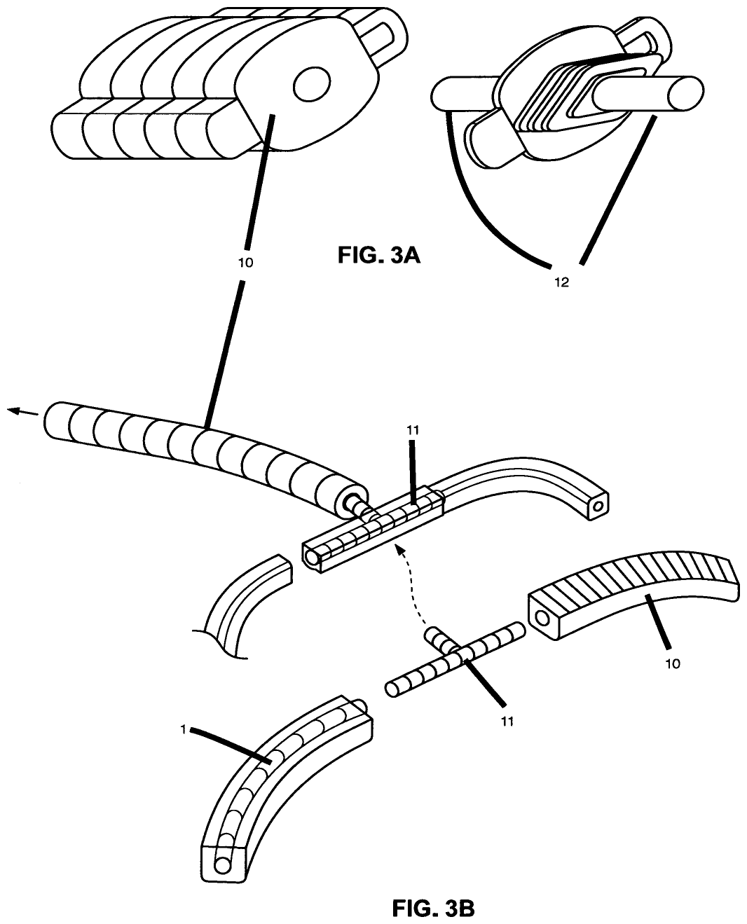

Referring to FIG. 3A the dual end oxygen connector (12) is a special part in joining the internal oxygen lines together within the Harness style systems. FIG. 3B shows the Tee connector which directs the flow of oxygen toward to the nose.

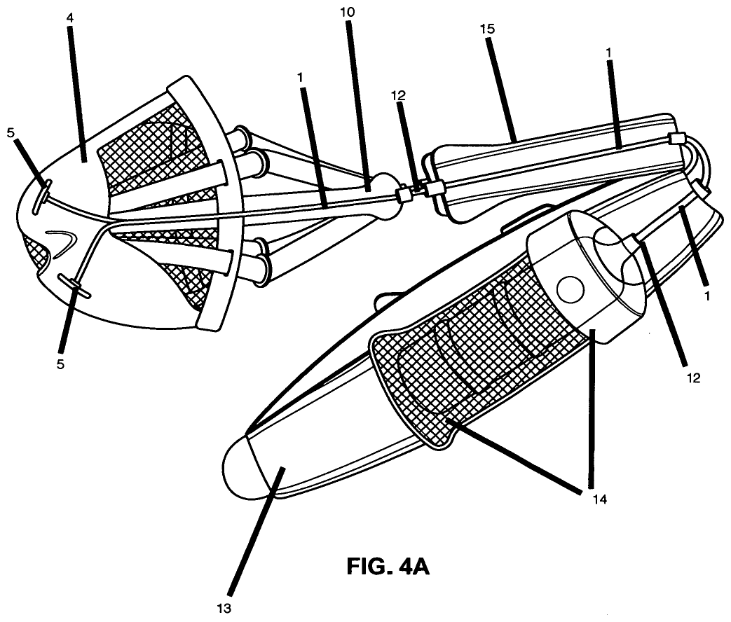

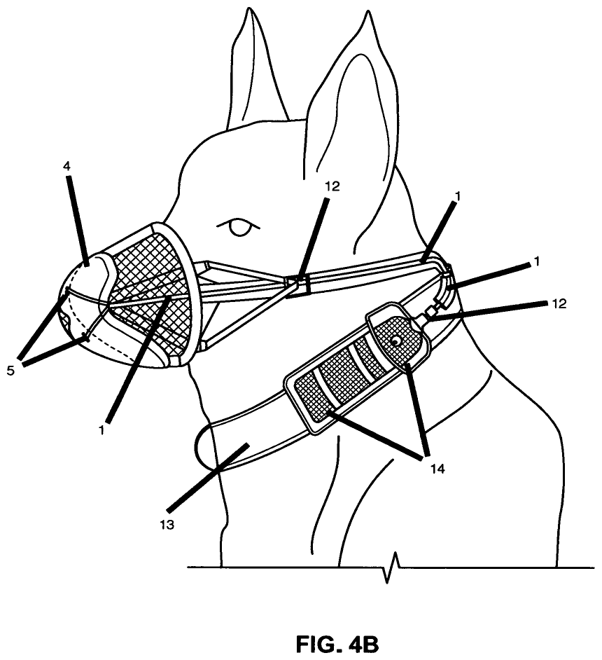

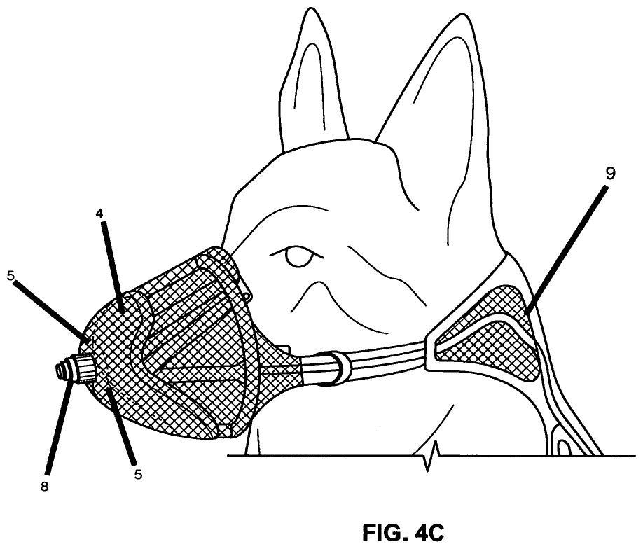

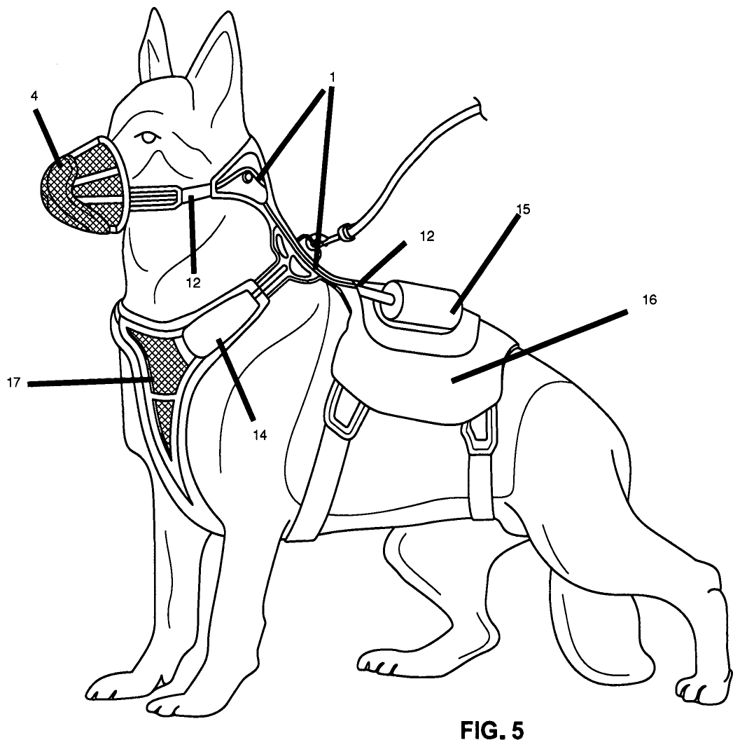

Referring to FIG. 4A shows the Full Neck Harness system. This will be worn by the canine which allows the carrying of their own oxygen. The fully integrated oxygen muzzle with oxygen tubing (1) and O2 ports (5) in the nose are connected by a special dual connector (12) to an oxygen bottle housed in the pouch (14) all built on a special collar (13) which holds the bottle. The rear straps (10) and (15) have the oxygen tubing (1) encased in flexible webbing for protection. Any connection or tube will be protected by being inside protective straps. These views are to see what is inside. FIG. 4B is the exact system as 4A but shown on the canine.

Looking at FIG. 4C shows the different placement of the Oxygen Port (8). It can be in the rear strap on either side toward the end as FIG. 1B showed or on the front of the nose. Both configurations have internal oxygen tubing directing the O2 to the nose and mouth of the canine and are secured behind the head with the heavy duty buckle (9).

This FIG. 5 depicts the canine wearing the Full Body Harness System. Everything is connected by the dual oxygen connectors (12) and all tubing completely encased and protected by flexible webbing and industrial stitched. All oxygen when in use runs from the back of the canine to the front nose where it is delivered through the O2 ports (5). The canine also has on a chest harness which is outfitted with more locations to attach more oxygen.

1. A soft style canine muzzle with fully integrated oxygen delivery capability
The specially designed oxygen connector on either left or right side
The internal oxygen tubing and how the oxygen flows
The oxygen port design that blows into nose or mouth of dog
